(19) United States
(12) Patent Application Publication
(10) Pub. No.: US 2018/0221108 A1
(43) Pub. Date: Aug. 9, 2018

(54) SURGICAL MARKER ELEMENT, SURGICAL REFERENCING UNIT, AND SURGICAL NAVIGATION SYSTEM

(71) Applicant: Aesculap AG, Tuttlingen (DE)

(72) Inventors: Holger Broers, Uplengen-Spols (DE); Andreas Göggelmann, Volkertshausen (DE); Tobias Pfeifer, Waldmössingen (DE)

(21) Appl. No.: 15/944,085

(22) Filed: Apr. 3, 2018

Related U.S. Application Data
(63) Continuation of application No. PCT/EP2016/074186, filed on Oct. 10, 2016.

(30) Foreign Application Priority Data
Oct. 9, 2015 (DE) 102015117239.9

(51) Int. Cl. A61B 90/00; A61B 34/20

(52) U.S. Cl. CPC A61B 90/39; A61B 34/20; A61B 2090/3937; A61B 2090/3983; A61B 2034/2055

(57) ABSTRACT

The invention relates to a marker element, in particular a medical or surgical marker element, for a referencing unit of a navigation system, which marker element is configured to be reflective to electromagnetic radiation, further comprising a layer comprising a multitude of retroreflective elements. An improved referencing unit and an improved navigation system are also provided.

### CROSS-REFERENCE TO RELATED APPLICATIONS

This application is a continuation of international application number PCT/EP2016/074186 filed on Oct. 10, 2016 and claims the benefit of German application number 10 2015 117 239.9 filed on Oct. 9, 2015, which are incorporated herein by reference in their entirety and for all purposes.

### FIELD OF THE INVENTION

The present invention relates to marker elements generally, and more specifically to a marker element, in particular a medical or surgical marker element, for a referencing unit of a navigation system, which marker element is configured to be reflective to electromagnetic radiation.

Further, the present invention relates to referencing units generally, and more specifically to a referencing unit, in particular a medical or surgical referencing unit, whose position and/or orientation in the room is detectable with a surgical navigation system, having at least one surgical marker element.

In addition, the present invention relates to navigation systems generally, and more specifically to a navigation system, in particular a medical or surgical navigation system, having at least one referencing unit comprising at least three marker elements and having at least one detection device for detecting the position and/or the orientation of the referencing unit in the room, wherein the referencing unit comprises at least one surgical marker element.

### BACKGROUND OF THE INVENTION

Marker elements, referencing units, and navigation systems in the form of surgical or medical marker elements, referencing units, and navigation systems are known from DE 10 2007 011 595 A1, for example. In particular, spheres that are covered with special films reflective to electromagnetic radiation, in particular infrared radiation, are thereby used as marker elements. For determining a position and/or orientation of the referencing unit in the room, the principle of triangulation is actualized with the navigation systems. Three-dimensional information from the referencing unit is calculated on the basis of angle measurements and a scale, for example of the orientation of two detectors in the form of cameras. Attributes of the referencing unit are thereby identified in the image data and are mapped. It is also known, depending on requirements, to apply artificial signalizations to the referencing unit and to measure them. A particularly good relationship between attributes of the referencing unit to be measured and a background present in an operating room, for example, is achieved by self-luminous marker elements, also designated as so-called active marker elements.

A problem in the known passive marker elements that are coated with special films is in particular that they only reflect diffusely, so that always only a small portion of the electromagnetic radiation sent out by the navigation system is sent back thereto. This is due in particular to the fact that in the case of a reflective surface, the radiation is only reflected back to the radiation source in orthogonal orientation.

### SUMMARY OF THE INVENTION

In a first aspect of the invention, a marker element is provided, in particular a medical or surgical marker element, for a referencing unit of a navigation system. Said marker element is configured to be reflective to electromagnetic radiation. Said marker element further comprises a layer comprising a multitude of retroreflective elements.

In a second aspect of the invention, a referencing unit is provided, in particular a medical or surgical referencing unit, whose position and/or orientation in the room is detectable with a surgical navigation system. Said referencing unit has at least one surgical marker element. Wherein the at least one marker element is configured to be reflective to electromagnetic radiation. Wherein the at least one marker element further comprises a layer comprising a multitude of retroreflective elements.

In a third aspect of the invention, a navigation system is provided, in particular a medical or surgical navigation system. Said navigation system has at least one referencing unit comprising at least three marker elements and at least one detection device for detecting at least one of the position and the orientation of the referencing unit in the room. Wherein the referencing unit comprises at least one surgical marker element. Wherein at least one of
a) the at least one marker element is configured to be reflective to electromagnetic radiation, wherein the at least one marker element further comprising a layer comprising a multitude of retroreflective elements
and
b) the referencing unit is designed in the form of a referencing unit whose at least one of position and orientation in the room is detectable with a surgical navigation system, which referencing unit has at least one surgical marker element, wherein the at least one marker element is configured to be reflective to electromagnetic radiation, and wherein the at least one marker element has a layer comprising a multitude of retroreflective elements.

### BRIEF DESCRIPTION OF THE DRAWING FIGURES

The foregoing summary and the following description may be better understood in conjunction with the drawing figures, of which:

FIG. 1: shows a schematic depiction of a navigation system;

FIG. 2: shows a perspective depiction of a referencing unit with four marker elements, of which one is depicted in a sectional view;

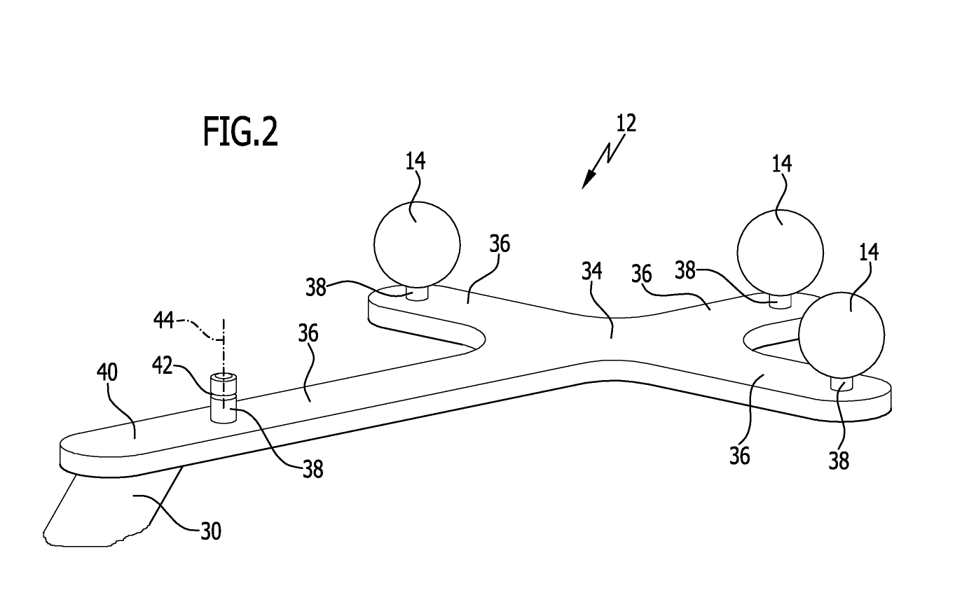

FIG. 3: shows a schematic depiction of the basic principle of retroreflection on a sphere;

FIG. 4: shows a schematic depiction of the beam path through a support made out of PMMA and provided with a layer of retroreflective elements;

FIG. 5: shows a schematic view of a hollow spherical shaped marker element with a layer of retroreflective elements which are arranged on an inner wall face of a hollow spherical shaped outer protective layer;

FIG. 6: shows a schematic depiction of the beam path when using a marker element from FIG. 5;

FIG. 7: shows a schematic sectional view of a two-part marker element with an outer protective/supporting layer;

FIG. 8: shows a schematic depiction of the beam path similar to FIG. 6, but in the case of a marker element as depicted in FIG. 7; and

FIG. 9: shows a schematic depiction of an intensity distribution of the radiation retroreflected by the sphere-shaped marker element depicted in FIG. 5.

### DETAILED DESCRIPTION OF THE INVENTION

Although the invention is illustrated and described herein with reference to specific embodiments, the invention is not intended to be limited to the details shown. Rather, various modifications may be made in the details within the scope and range of equivalents of the claims and without departing from the invention.

The present invention relates to a marker element, in particular a medical or surgical marker element, for a referencing unit of a navigation system, which marker element is configured to be reflective to electromagnetic radiation, further comprising a layer comprising a multitude of retroreflective elements.

The solution proposed in accordance with the invention allows in particular for electromagnetic radiation impinging on the marker element to be substantially entirely reflected back, i.e. retroreflected. The electromagnetic radiation is therefore reflected back substantially in parallel in the direction from which it impinges the marker element and in particular on its retroreflective elements. In addition, it is not absolutely necessary for all retroreflective elements, in particular if they are configured in the form of spheres, to have the exact same diameter. This does not matter, in particular because each individual retroreflective element again reflects back the incident light in the same direction. It thereby does not depend on the size of the retroreflective element.

The marker element may be produced in a particularly simple manner if the retroreflective elements are configured in the form of spheres. Spheres, in particular glass spheres, may be manufactured in large quantity and highly reproducibly. Incident light is reflected back by the spheres in parallel to the direction of incidence.

In order to be able to also design marker elements with small diameters to be retroreflective, it is favorable if the spheres have a diameter in a range of about 10 μm to about 50 μm. In particular, it is favorable if the spheres have a diameter of about 20 μm.

Advantageously, the retroreflective elements are made of glass or a plastics material. Spheres in particular may be made of glass and plastics material in high quantity and precision.

It is particularly favorable if the material out of which the retroreflective elements are made has a refractive index with a value in a range of about 1.5 to about 2.5, in particular with a value of about 1.93, or a refractive index with a value in a range of about 2.5 to about 3.4, in particular with a value of about 2.9. A value of the refractive index in the specified ranges is favorable in particular if, as subsequently proposed, the retroreflective elements are provided with a protective layer. In this case, it is advantageous if the refractive index of the retroreflective elements is matched to the refractive index of the coating in order to achieve the desired retroreflection, despite the present protective layer.

Marker elements may be particularly cost-effectively and efficiently produced if the layer of the multitude of retroreflective elements is formed as a single layer. In particular, it is favorable if the outer surface of the marker element is fully provided with a single-ply layer of retroreflective elements. In this way, the entire surface of the marker element may send a largest possible portion of incident electromagnetic radiation back in the direction from which it impinges on the marker element.

In order to obtain a best possible retroreflection, it is advantageous if the multitude of retroreflective elements is each at least partially provided with a coating reflective to electromagnetic radiation. Similar to a mirror, retroreflective elements out of glass that are provided with the specified coating, for example on a backside thereof, may reflect back incident electromagnetic radiation in parallel to the direction of incidence. The electromagnetic radiation is then reflected in particular at the boundary layer between the retroreflective element and the coating.

The marker element may be produced in a particularly simple manner if the coating is formed out of a metal. For example, the coating may be produced by vapor deposition.

A marker element has particularly good retroreflective properties if the metal is silver or aluminum. In particular coatings out of aluminum may be produced in a simple and cost-effective manner.

For the production and handling of the marker element, it is advantageous if the marker element comprises a support for the layer of retroreflective elements. The support serves in particular on the one hand to bear the multitude of retroreflective elements. On the other hand, it acts in particular also as a protective layer for the retroreflective elements.

Preferably, the support is formed out of a support material permeable to electromagnetic radiation. In particular, it is favorable if the support material does not allow the adhesion of liquids and/or other contaminants to as great an extent as possible. It may thus be prevented that practically only a part of the marker element is “visible” to the navigation system if debris covers a part of an outer surface of the marker element. The permeability of the support material to electromagnetic radiation allows in particular for the electromagnetic radiation used for the navigation of the referencing unit to be able to penetrate the support as unhindered as possible, to reach the retroreflective elements, and to be reflected back thereby in the direction of incidence or in parallel thereto.

The marker element may be produced in a particularly simple and cost-effective manner if the support material is glass or plastics material. These materials may in particular be constructed to be permeable to electromagnetic radiation. The use of a support material out of plastics material has in particular the advantage that a deformation of the support may optionally still be possible after applying the layer of retroreflective elements, for example if the plastics material is a thermoplastic plastics material.

Favorably, the plastics material is or contains polymethylmethacrylate (PMMA). This plastics material, also designated as acrylic glass, is permeable to electromagnetic radiation. In addition, it may be produced in a simple and cost-effective manner. The coating of the support out of PMMA with the retroreflective elements may occur in particular then when the plastics material is not fully hardened, so that the retroreflective elements may adhere on its surface or even be partially embedded into the support.

In accordance with another preferred embodiment, provision may be made for the support material to have a support material refractive index which is less than the refractive index of the retroreflective elements. This allows in particular for electromagnetic radiation impinging on the marker element to be able to get through the support and impinge on the retroreflective elements.

Preferably, the support material refractive index has a value in a range of about 1.3 to about 1.7. In particular, the support material refractive index may have a value of about 1.5. For example, the refractive index of PMMA has a value of about 1.49, such that said plastics material is superbly suitable as support material.

In order to obtain a marker element that is as stable as possible, it is advantageous if the layer of retroreflective elements is at least partially embedded into the support. Thus, a particularly good adhesion of the retroreflective elements to the support may be achieved.

The production of the marker element is further simplified if the reflective coating is applied to the layer of retroreflective elements which is at least partially embedded into the support. This may be achieved in particular by, in the production of the marker element, first providing the support with the layer of retroreflective elements and only thereafter applying the coating reflective to electromagnetic radiation to the layer of retroreflective elements.

In general, marker elements may have any form. It is favorable in particular if the layer of retroreflective elements is planar or substantially planar or defines a section of an ellipse surface or a sphere surface. Thus, in particular planar, i.e. flat markers element may be formed or also sphere-shaped or substantially sphere-shaped marker elements. In particular sphere-shaped marker elements have the advantage that they always show a substantially circular viewing face for the navigation system, practically independently of an orientation in the room.

Preferably, the marker element is formed elliptical or sphere-shaped or substantially sphere-shaped. As already outlined, it thus practically does not matter for the navigation of the referencing unit how the marker element is oriented in the room. The navigation system thus substantially always sees a circular face.

In accordance with another preferred embodiment, provision may be made for the reflective coating to point in the direction or substantially in the direction toward a midpoint of the sphere-shaped or substantially sphere-shaped marker element. This means in particular that the support points away from the midpoint of the marker element and thus forms an outer layer or coating protecting the retroreflective elements.

In particular, it is favorable if an outer surface of the marker element is formed by the support. The support may thus protect the retroreflective elements from contaminants, in particular from contaminants that otherwise could penetrate into the interspaces between the retroreflective elements and adhere there.

For the production of the marker element, it may be advantageous if it is formed in two or more parts. In particular, it may consist of two or more marker element parts. For example, a sphere-shaped marker element may thus be produced in a simple manner.

It is favorable if the two or more marker element parts are configured in the form of a half-shell or substantially in the form of a half-shell. In particular sphere-shaped marker elements out of two marker element parts in the form of half-shells may thus be constructed. For example, planar or half-shell-shaped supports may be formed, whose inner faces are coated with the retroreflective marker elements. The marker element parts are then assembled to form the marker element.

Preferably, the retroreflective coating delimits an ellipsoidal or spherical or substantially spherical cavity. Very light marker elements, for example, may thus be formed. In particular due to the coating reflective to electromagnetic radiation, which coating may point in the direction toward a midpoint of the cavity, it is not absolutely necessary to fill said cavity.

A stability of the marker element may be improved in particular by filling the cavity with a filling material.

The marker element may be produced in a particularly simple and cost-effective manner if the filling material is a plastics material. In particular, it may be a sterilizable material in the case of the plastics material. For example, the filling material may already be produced in a form that corresponds to a form or shape of the cavity so that, in particular if the marker element comprises multiple marker element parts, these may be slipped over the filling material. The filling material may, however, also be filled into the cavity only upon assembling the marker element parts.

The present invention further relates to a referencing unit, in particular medical or surgical referencing unit, whose position and/or orientation in the room is detectable with a surgical navigation system, having at least one surgical marker element, wherein the at least one marker element is configured to be reflective to electromagnetic radiation, wherein the at least one marker element further comprising a layer comprising a multitude of retroreflective elements.

Equipping the referencing unit with marker elements in accordance with the invention enables in particular an improved visibility of the referencing unit in the room and thereby a more precise determination by the navigation system of position and/or orientation thereof in the room.

It is favorable for the handleability of the referencing unit if it comprises a support on which the at least one marker element is arranged or formed. For example, conventional supports may be used here with coupling devices for detachably connecting to in each case one marker element. The marker elements may then be formed as disposable marker elements, for example, the support may be cleaned and used again after sterilization. Of course, the marker elements may also be designed to be treatable in order to be used multiple times. Prerequisite is then merely that the materials out of which the marker elements are formed are in particular resistant against alkaline cleaners and withstand undergoing at least one superheated steam sterilization cycle.

The present invention further relates to a navigation system, in particular a medical or surgical navigation system, having at least one referencing unit comprising at least three marker elements and having at least one detection device for detecting the position and/or the orientation of the referencing unit in the room, wherein the referencing unit comprises at least one surgical marker element, wherein at least one of
a) the at least one marker element is configured to be reflective to electromagnetic radiation, wherein the at least one marker element further comprising a layer comprising a multitude of retroreflective elements
and
b) the referencing unit is designed in the form of a referencing unit whose at least one of position and orientation in the room is detectable with a surgical navigation system, which referencing unit has at least one surgical marker element, wherein the at least one marker element is configured to be reflective to electromagnetic radiation, and wherein the at least one marker element has a layer comprising a multitude of retroreflective elements.

Equipping a navigation system with such marker elements and referencing units, respectively, has the advantage that the visibility of the marker elements and of the referencing units is significantly improved by the detecting device of the navigation system, in comparison to conventional marker elements with diffusely reflective surfaces.

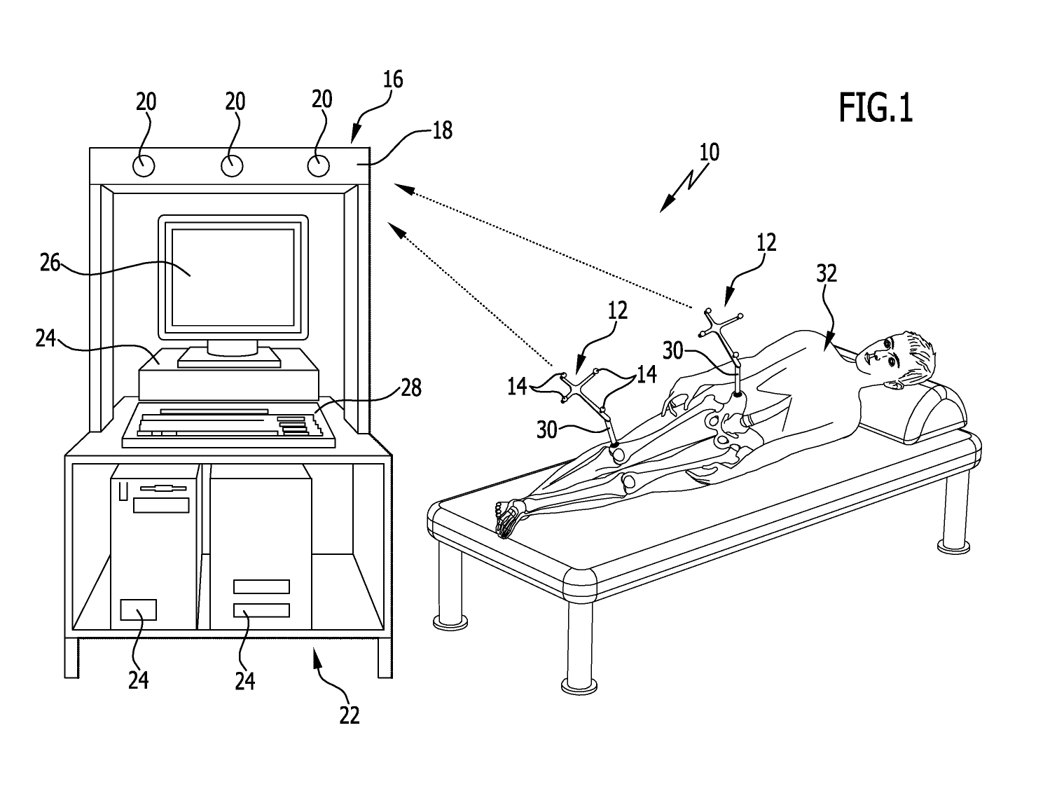

A navigation system provided as a whole with the reference numeral 10 is depicted for example in FIG. 1. It comprises multiple referencing units 12 which comprise preferably three, in the embodiments depicted in the Figures in each case four, marker elements 14.

The navigation system 10 comprises a sending and receiving unit 16 for emitting and receiving electromagnetic radiation and/or ultrasound. It comprises a beam-shaped support 18 on which three senders/receivers 20 are arranged, with which electromagnetic radiation and ultrasound, respectively, may be emitted and/or received. In principle, only two senders/receivers 20 could be provided. In order to improve accuracy in the determination of position of the referencing units 12, three or more senders/receivers 20 of that kind may also be provided. Moreover, the navigation system 10 comprises a data processing system 22 which, in the embodiment depicted in FIG. 1, comprises three computers 24 connected together, a monitor 26, and an input device in the form of a keyboard 28. Signals produced and/or received by the sending and receiving unit 16 may be processed with the data processing system 22 in order to determine a position and/or an orientation of a referencing unit 12 in the room.

Referencing units 12 may in particular be formed in such a way that they may be fixed, with the corresponding adapters 30, to a patient 32, for example. In particular, joint positions and joint centers of the patient 32 may thus be determined by moving a body part of the patient 32, to which a first referencing element 12 is fixed, relative to another body part of the patient 32, to which a further referencing unit 12 is fixed. Alternatively, a referencing unit 12 may also be arranged on a surgical instrument or a tool, for example by using an adapter 30 suitable therefor.

The referencing unit 12 comprises a cross-shaped support 34 which comprises four support arms 36 arranged substantially perpendicular relative to each other, which support arms 36 may in particular have different lengths. Each support arm 36 bears a connecting element in the form of a pin-shaped adapter 38 which each project perpendicularly out from a planar surface 40 of the support 34. Each adapter 38 is provided with a circumferential annular groove 42 which forms a latching element. The annular groove 42 is arranged concentrically to a longitudinal axis 44 defined by the adapter 38 and divides the adapter 38 into two parts that are at a length ratio of about two to three, wherein the longer part of the adapter 38 directly adjoins the support 34.

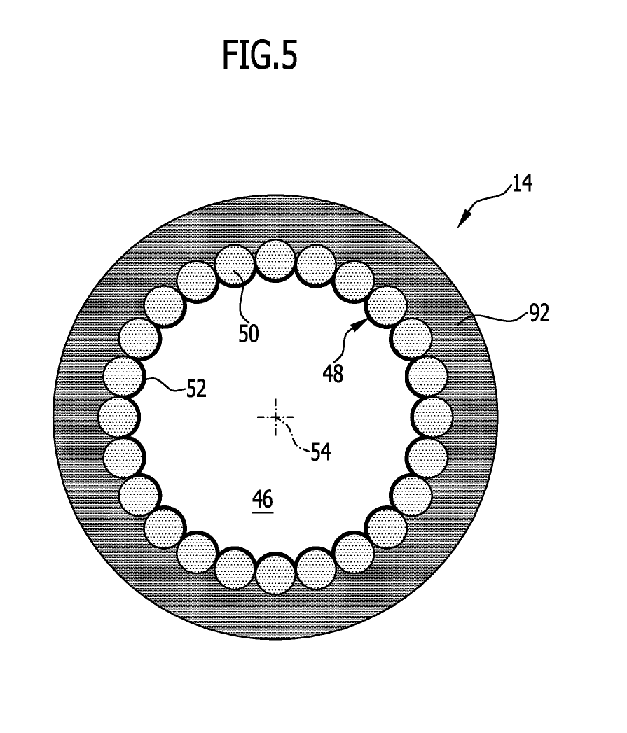

The construction of a first embodiment of one of the marker elements 14 is schematically depicted in FIG. 5. It comprises a support 92 configured in the form of a spherical shell, which support 92 surrounds a hollow core 46. The support 92 is provided on an inner side facing the midpoint 54 of the spherical shell with a multitude of retroreflective elements 48 in the form of spheres 50 out of plastics material or glass.

An outer surface of the spheres 50 is partially provided with a coating 52 reflective to electromagnetic radiation. The coating 52 covers half of the outer surface of each sphere 50, namely in such a way that the outer surfaces of the spheres 50 are provided with the coating 52 substantially only on their surface regions adjoining the hollow core. The spheres 50 with the coating 52 are therefore arranged in such a way that the coated half-spherical surfaces point in the direction toward a midpoint 54 of the core 46.

The retroreflective elements 48 may be mounted directly to the inner side and to the inner surface of the support 92, respectively, with a glue or adhesive. They may also optionally be partially embedded into the support 92.

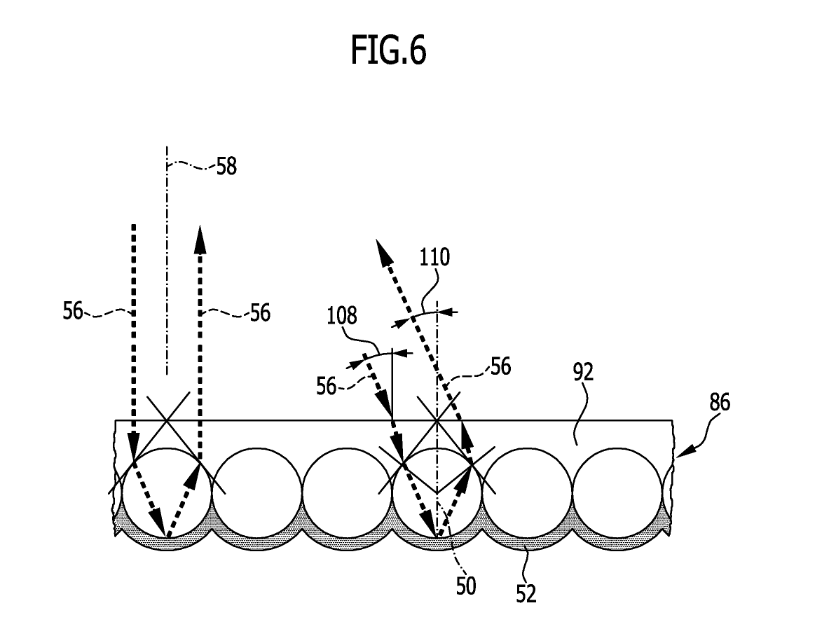

A beam path of electromagnetic radiation 56 impinging on the marker element 14 is schematically depicted in FIG. 6. A transition from an optically thinner to an optically thicker medium occurs here already upon the radiation impinging on the support 92. If the radiation 56 does not impinge on the support 92 perpendicularly, then it is refracted to the perpendicular already upon the transition from air into the support 92.

The refractive index of the spheres 50 is preferably selected such that it is greater than a refractive index of the support material out of which the support 92 is produced. For example if the support 92 is formed out of polymethylmethacrylate (PMMA), whose refractive index is about 1.49, it is advantageous if a refractive index of the spheres 50 is about 2.9. This may be achieved by correspondingly selecting a plastics material or a glass for producing the spheres 50. By means of this selection of the refractive indices, a further transition arises from an optically thinner medium, namely the support 92, into an optically thicker medium, namely the spheres 50. The radiation 56 is again refracted to the perpendicular.

Each sphere 50 may be formed multi-layered or may also consist of a material mixture. These embodiments enable in particular an individual adjustment of the refractive index of the spheres 50.

The radiation 56 is reflected at the boundary layer between the sphere 50 and the coating 52. Further, the radiation 56 changes its direction again upon exiting the sphere 50 into the support 92 and upon exiting the support 92 into the surrounding air, such that is sent back again to the sender/receiver 20 of the navigation system 10 in parallel to a direction of incidence 58 to which it also impinged on the sphere 50 in parallel.

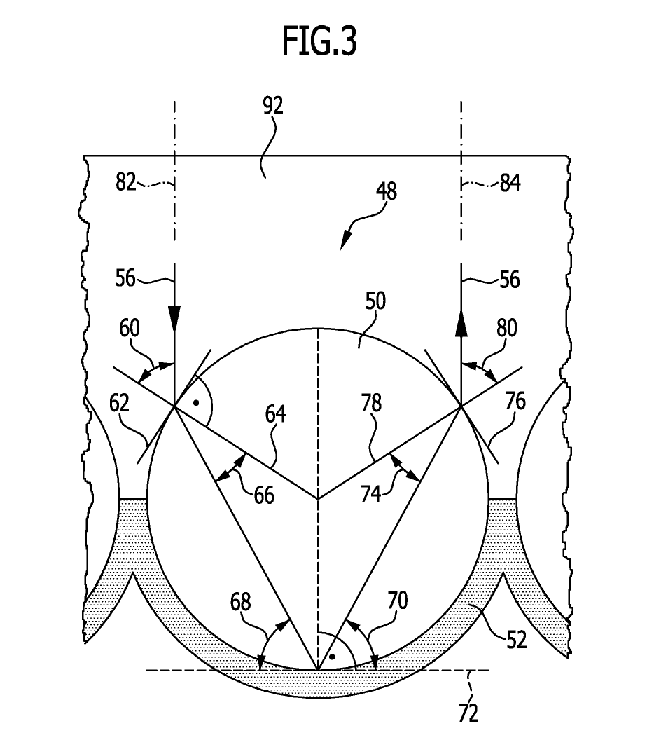

The beam path from FIG. 6 is depicted again in FIG. 3 in detail on a sphere 50. The radiation 56 impinging perpendicularly on the support 92 impinges on the sphere 50 at an angle of incidence 60 with respect to a tangential plane 62 of the sphere 50 and is refracted to the face normal 64 standing perpendicularly to the tangential plane 62. An angle of reflection 66 in the optically thicker medium is greater than the angle of incidence 60.

The radiation 56 is totally reflected on a boundary layer between the sphere 50 and the coating 52. It applies here that an angle of incidence 68 coincides with the angle of reflection 70, wherein the reflection occurs at the boundary layer between sphere 50 and coating 52 with respect to a tangential plane 72.

Because the sphere 50 is fully symmetrical, the radiation 56 again impinges on a boundary face between the sphere 50 and the support at an angle of reflection 74 which coincides with the angle of reflection 66. With respect to a face normal 78 running perpendicularly to a tangential plane 76 in the exit point of the radiation 56 out of the sphere 50, the radiation 56 exits the sphere 50 at an angle of incidence 80, wherein the angle of incidence 80 corresponds to the angle of incidence 60. A direction of incidence 82 of the radiation 56 thus runs parallel to a direction of reflection 84 of the totally reflected radiation 56.

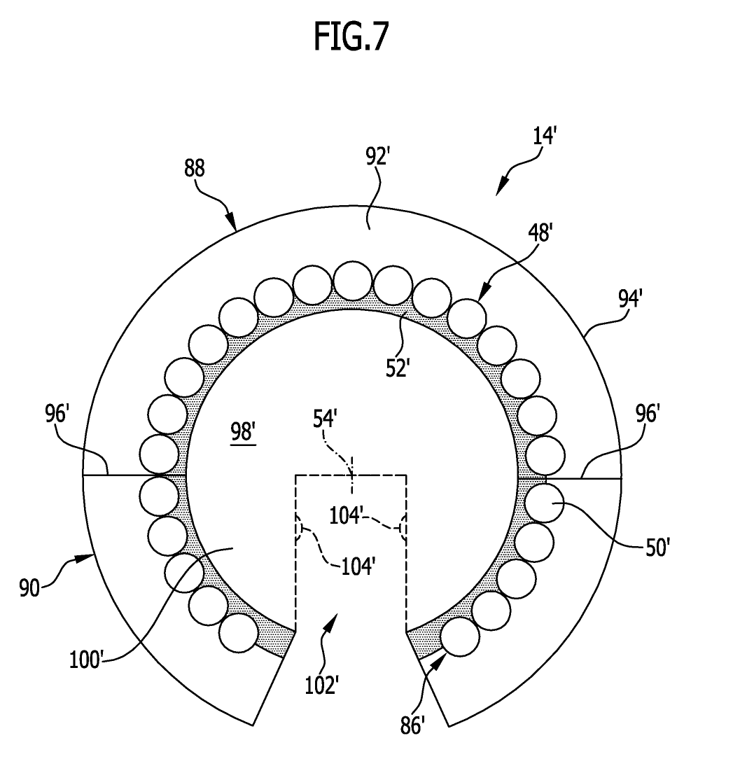

A second embodiment of a marker element designated as a whole with the reference numeral 14′ is depicted schematically in FIG. 7. Similarly to the marker element 14 depicted in FIG. 5, the size ratios between the retroreflective elements 48 and 48′, respectively, do not coincide with the dimensions of the marker elements 14 and 14′. The marker elements 14 and 14′ may for example have a diameter of about 10 mm, the retroreflective elements 48 and 48′ may have a diameter of about 20 μm. This results in a layer 86 and 86′, respectively, of the retroreflective elements 48 and 48′, respectively, comprising significantly more spheres 50 and 50′, respectively, than is depicted in the figures.

The marker element 14′ is formed in two parts and comprises a first marker element part 88′ and a second marker element part 90′ that are each formed substantially like a half-shell. Each marker element part 88′ comprises a support 92′ permeable to an electromagnetic radiation, which has an outer surface 94′ that points away from a midpoint 54′ of the marker element 14′. The surface 94′ is thus convexly curved pointing away from the marker element 14′.

The spheres 50′ are partially embedded into the support 92′. In the embodiment of the marker element 14′ depicted in FIG. 7, by about half.

The layer 86′ of the retroreflective elements 48′ is formed as a single layer and points in the direction of the midpoint 54′ with its side pointing away from the support 92′. Further, the spheres 50′ are, in turn, provided with a coating 52′ reflective to electromagnetic radiation. This forms an inner surface of the marker element parts 88′ and 90′ defining hemispheres. The marker element parts 88′ and 90′ are materially bonded to each other with a layer of adhesive 96′.

Optionally, a spherical cavity 98′ delimited by the coating 52′ may be filled with a filling material. This preferably forms the shape of a sphere 100′ with a coupling recess 102′ which is preferably formed corresponding to the adapter 38.

In particular projecting latching elements 104′ may be provided at the coupling recess 102′, the latching elements 104′ engaging in the annular groove 42 when the marker element 14′ is coupled to the adapter 38.

The sphere 100′ is preferably formed out of a sterilizable plastics material. In particular, the marker element parts 88′ and 90′ are materially bonded to the sphere 100′, for example by means of a layer of adhesive.

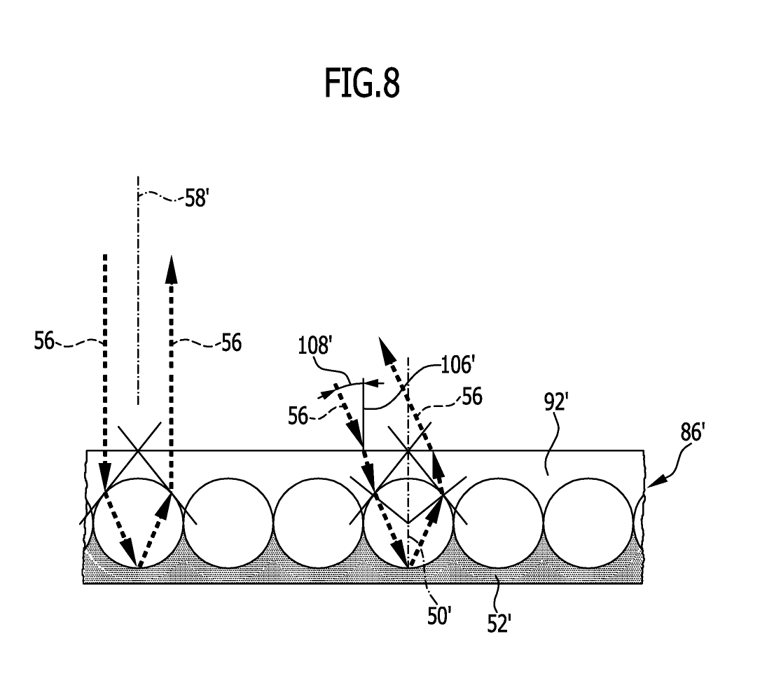

A beam path of electromagnetic radiation 56 impinging on the marker element 14′ is depicted schematically in FIG. 8. Hereby, a transition from an optically thinner to an optically thicker medium already occurs upon the radiation impinging on the support 92′.

The refractive index of the spheres 50′ is preferably selected such that it is greater than a refractive index of the support material out of which the support 92′ is produced. For example, if the support 92′ is made out of polymethylmethacrylate (PMMA), the refractive index of which is about 1.49, it is advantageous if a refractive index of the spheres 50′ is about 2.9. This may be achieved by correspondingly selecting a plastics material or a glass for forming the spheres 50′.

Each sphere 50′ may, as well as each sphere 50, be formed in multiple layers or may also consist of a material mixture. These embodiments enable in particular an individual adjustment of the refractive index of the spheres 50 and 50′.

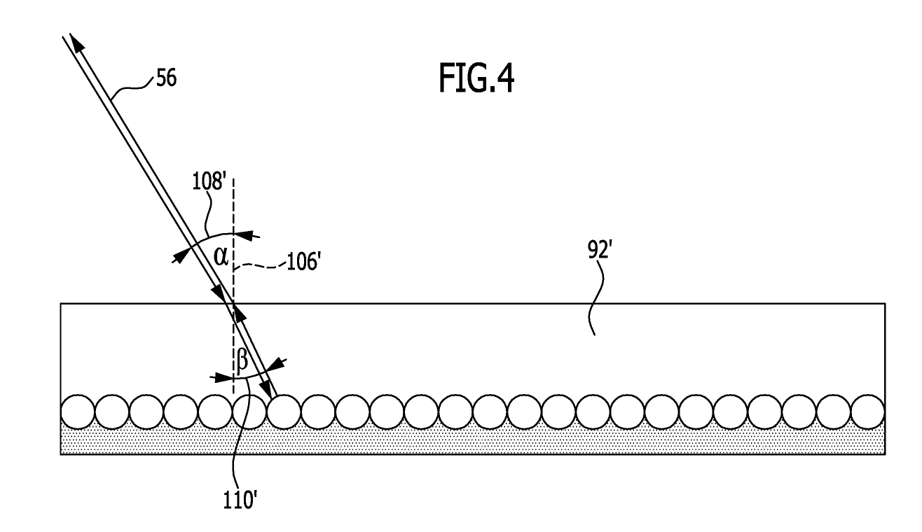

As may be discerned from FIGS. 4 and 8, the electromagnetic radiation 56 is refracted toward the face normal 106′ of an outer surface of the support 92′ upon entering into the support 92′. An angle of incidence 108′ is greater than an angle of reflection 110′ in the optically thicker support 92′. The beam path of the electromagnetic radiation 56 upon the transition from the support 92′ into the sphere 50′ corresponds substantially to the path as depicted in FIG. 3, because the support 92′ is produced out of an optically thinner material than the spheres 50′, such that the law of refraction described in conjunction with FIG. 3 applies here by analogy.

The support 92′ allows for a retroreflection that is as free of interference as possible, because a contamination of an outer surface of the support 92′, for example due to fat or water, then leads merely to a parallel offset and not to a massive disruption in the visibility of the marker element 14′, as is the case in conventional marker elements.

The production of the marker element parts 88 and 90′ may occur by first providing a planar support 92′ with the layer 86′ of retroreflective elements 48′. A plate out of acrylic glass thus provided for example with embedded spheres 50′ may then be formed to a half hollow sphere in a next step, as is depicted for example in section in FIG. 7 as marker element part 88′ and 90′, respectively.

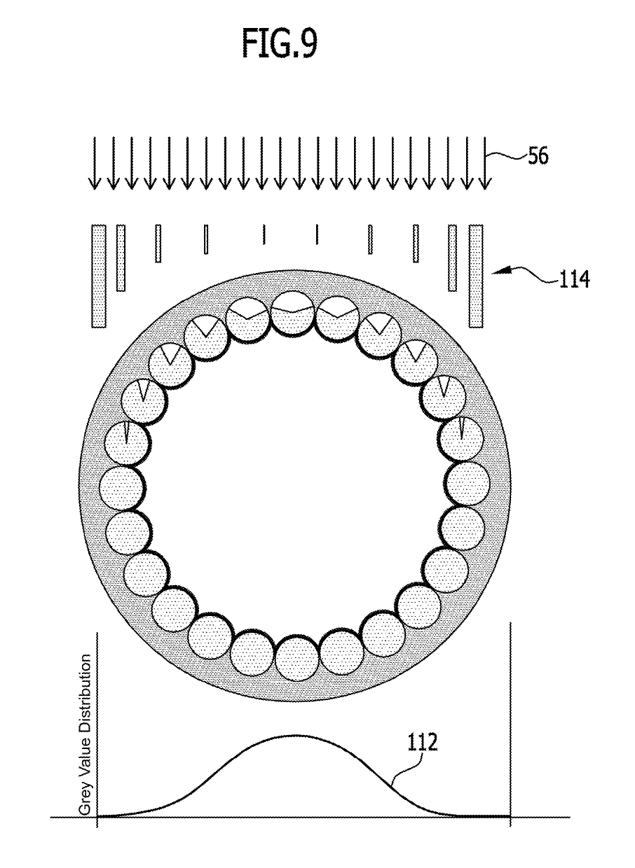

An intensity distribution 112 of the radiation retroreflected by a marker element 14 and 14′, respectively, is depicted schematically in FIG. 9, as it arises from an image of the marker element 14 and 14′, respectively, taken with the sending and receiving unit 16 of the navigation system 10. Because only a portion of the radiation 56 may be retroreflected by the retroreflective elements 48 and 48′, respectively, upon incidence of radiation 56 on the marker element 14 and 14′, respectively, taking into account the refractive indices of the spheres 50 and 50′, respectively, and of the support 92 and 92′, respectively, a significantly weakened intensity signal arises in the outer region of the image of the marker elements 14 and 14′, respectively. The not-reflected portions 114 of the radiation 56 are symbolized above the schematically depicted marker element as dotted bars, for example.

Overall, one obtains with the marker elements 14 and 14′ an intensity or grey value distribution with a maximum which, in the case of an uncontaminated marker element 14 and 14′, respectively, indicates a midpoint of the marker element 14 and 14′, respectively. This is due to the fact that in each case only a part of the sphere 50 and 50′, respectively, may even send back the incident radiation when forming the marker elements 14 and 14′, respectively, in the described way.

### REFERENCE NUMERAL LIST

10 Navigation system
12 referencing unit
14 marker element
16 sending and receiving unit
18 support
20 sender/receiver
22 data processing system
24 computer
26 monitor
28 keyboard
30 adapter
32 patient
34 support
36 support arm
38 adapter
40 surface
42 annular groove
44 longitudinal axis
46 core
48, 48′ retroreflective element
50, 50′ sphere
52, 52′ coating
54, 54′ midpoint
56 radiation
58, 58′ direction of incidence
60 angle of incidence
62 tangential plane
64 face normal
66 angle of reflection
68 angle of incidence
70 angle of reflection
72 tangential plane
74 angle of reflection
76 tangential plane
78 face normal
80 angle of incidence
82 direction of incidence
84 direction of reflection
86, 86′ layer
88′ marker element part
90′ marker element part
92, 92′ support
94′ surface
96′ layer of adhesive
98′ cavity
100′ sphere
102′ coupling recess
104′ latching element
106′ face normal
108, 108′ angle of incidence
110, 110′ angle of reflection
112 intensity distribution
114 not-reflected portions

1. A marker element, in particular a medical or surgical marker element, for a referencing unit of a navigation system, which marker element is configured to be reflective to electromagnetic radiation, further comprising a layer comprising a multitude of retroreflective elements.

2. The marker element in accordance with claim 1, wherein the retroreflective elements are constructed in the shape of spheres.

3. The marker element in accordance with claim 2, wherein the spheres have a diameter in a range of about 10 μm to about 50 μm, in particular a diameter of about 20 μm.

4. The marker element in accordance with claim 1, wherein the retroreflective elements are made of glass or a plastics material.

5. The marker element in accordance with claim 1, wherein the material out of which the retroreflective elements are made has a refractive index with a value in a range of about 1.5 to about 2.5, in particular with a value of about 1.93, or a refractive index with a value in a range of about 2.5 to about 3.4, in particular with a value of about 2.9.

6. The marker element in accordance with claim 1, wherein the layer of the multitude of retroreflective elements is formed as a single layer.

7. The marker element in accordance with claim 1, wherein the multitude of retroreflective elements is each at least partially provided with a coating reflective to electromagnetic radiation.

8. The marker element in accordance with claim 7, wherein at least one of:
a) the coating is formed out of a metal, in particular by vapor deposition,
wherein, in particular, the metal is silver or aluminum,
and
b) the reflective coating delimits an ellipsoidal or spherical or substantially spherical cavity.

9. The marker element in accordance with claim 1, further comprising a support for the layer of retroreflective elements.

10. The marker element in accordance with claim 9, wherein at least one of:
a) the support is formed out of a support material permeable to electromagnetic radiation,
and
b) an outer surface of the marker element is formed by the support.

11. The marker element in accordance with claim 10, wherein at least one of:
a) the support material is glass or plastics material,
wherein, in particular, the plastics material is or contains polymethylmethacrylate (PMMA),
and
b) the support material has a support material refractive index which is less than the refractive index of the retroreflective elements,
wherein, in particular, the support material refractive index has a value in a range of about 1.3 to about 1.7, in particular a value of about 1.5.

12. The marker element in accordance with claim 9, wherein at least one of:
a) the layer of retroreflective elements is at least partially embedded into the support
and
b) the reflective coating is applied to the layer of retroreflective elements which is at least partially embedded into the support.

13. The marker element in accordance with claim 1, wherein at least one of:
a) the layer of retroreflective elements is planar or substantially planar or defines a section of an ellipse surface or a sphere surface,
and
b) the marker element is elliptical or sphere-shaped or substantially sphere-shaped,
wherein, in particular, the reflective coating points in the direction or substantially in the direction toward a midpoint of the sphere-shaped or substantially sphere-shaped marker element.

14. The marker element in accordance with claim 1, wherein the marker element is formed in two or more parts, in particular out of two or more marker element parts.

15. The marker element in accordance with claim 14, wherein the two or more marker element parts are configured in the form of a half-shell or substantially in the shape of a half-shell.

16. The marker element in accordance with claim 15, wherein the cavity is filled with a filling material.

17. The marker element in accordance with claim 16, wherein the filling material is a plastics material, in particular a sterilizable plastics material.

18. A referencing unit, in particular medical or surgical referencing unit, whose position and/or orientation in the room is detectable with a surgical navigation system, having at least one surgical marker element, wherein the at least one marker element is configured to be reflective to electromagnetic radiation, wherein the at least one marker element further comprising a layer comprising a multitude of retroreflective elements.

19. The referencing unit in accordance with claim 18, further comprising a support on which the at least one marker element is arranged or formed.

20. A navigation system, in particular a medical or surgical navigation system, having at least one referencing unit comprising at least three marker elements and having at least one detection device for detecting the position and/or the orientation of the referencing unit in the room, wherein the referencing unit comprises at least one surgical marker element, wherein at least one of:
a) the at least one marker element is configured to be reflective to electromagnetic radiation, wherein the at least one marker element further comprising a layer comprising a multitude of retroreflective elements,
and
b) the referencing unit is designed in the form of a referencing unit in whose at least one of position and orientation in the room is detectable with a surgical navigation system, which referencing unit has at least one surgical marker element, wherein the at least one marker element is configured to be reflective to electromagnetic radiation, and wherein the at least one marker element has a layer comprising a multitude of retroreflective elements.
